(19) United States
(12) Patent Application Publication
(10) Pub. No.: US 2023/0414348 A1
(43) Pub. Date: Dec. 28, 2023

(54) SYSTEMS, DEVICES, AND METHODS FOR IMPLANTABLE VALVE SKIRTS

(71) Applicant: EDWARDS LIFESCIENCES CORPORATION, Irvine, CA (US)

(72) Inventors: David Maimon, Atlit (IL); Melissa Fusari, Boise, ID (US); Tamir S. Levi, Zikhron Yaakov (IL)

(21) Appl. No.: 18/459,319

(22) Filed: Aug. 31, 2023

Related U.S. Application Data
(63) Continuation of application No. PCT/US22/19690, filed on Mar. 10, 2022.
(60) Provisional application No. 63/254,815, filed on Oct. 12, 2021.
(60) Provisional application No. 63/159,918, filed on Mar. 11, 2021.

(51) Int. Cl. A61F 2/24

(52) U.S. Cl. CPC A61F 2/2418; A61F 2230/0069

(57) ABSTRACT

An implantable prosthetic valve may comprise a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level. A plurality of prosthetic valve leaflets may be positioned within the flow channel and extend inward from the inner surface of the valve body.

### CROSS-REFERENCE TO RELATED APPLICATIONS

This application is a continuation of PCT patent application no. PCT/US2022/019690 filed on Mar. 10, 2022, which application claims the benefit of U.S. Provisional Application No. 63/254,815, filed Oct. 12, 2021, and U.S. Provisional Application No. 63/159,918, filed Mar. 11, 2021, the entire contents of each of which are incorporated herein by this specific reference.

### BACKGROUND

### Field

Certain embodiments disclosed herein relate generally to implantable medical devices and may relate to implantable prosthetic valves such as those which may be used in place of native cardiac valves.

### Background

A variety of maladies may affect an individual's body. Such maladies may be of the individual's heart, and may include maladies of the individual's native heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a patient's heart. Prosthetic implants, such as prosthetic valves, may be provided to replace or repair a portion of a patient's heart. Prosthetic aortic, mitral, tricuspid, and even pulmonary valves may be provided.

Implants may be deployed to the desired portion of the patient's body percutaneously, in a minimally invasive manner. Such deployment may occur transcatheter, in which a catheter may be deployed through the vasculature of an individual.

During deployment of such implants to a native heart valve for example, the native heart valve leaflets may remain within the patient's body. The native leaflets may be pushed aside by the deployment of the prosthetic valve, with the prosthetic leaflets performing the function previously provided by the native leaflets. Implanted prosthetic valves might include a skirt. In some cases, explant of the prosthetic valve is required, in which case the implanted prosthetic valve is surgically removed from the patient's body. However, explant of the previously implanted valve may prove to be challenging and risky where tissues may have formed between the skirt and the surrounding anatomy. In such cases, the surrounding tissues may be surgically cut away. Such surgical procedures for cutting away these tissues may be extremely delicate and may entail risks to the patent. It may be desirable to improve features of an implantable medical device, including an implantable prosthetic valve, for implantation within a patient's body, for functioning within a patient's body, or for removal from a patient's body.

### SUMMARY

Features of embodiments herein may be directed to improved features of medical devices for implantation within a patient's body, for functioning within a patient's body, or for removal from a patient's body. Features of embodiments herein may be directed to features of skirts that allow for improved sealing, and may allow for improved removal or explant procedures of a prosthetic valve. Features of embodiments herein may allow for reduced trauma to a patient's body during a removal or explant procedure, due to a reduced need for cutting of native tissue during a removal or explant procedure.

In embodiments herein, an implantable prosthetic valve is disclosed, including a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level. A plurality of prosthetic valve leaflets may be positioned within the flow channel and may extend inward from the inner surface of the valve body.

In embodiments herein, a method is disclosed, including implanting a prosthetic valve within a patient's body. The prosthetic valve may include a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level. A plurality of prosthetic valve leaflets may be positioned within the flow channel and extend inward from the inner surface of the valve body.

In embodiments herein, a method is disclosed, including removing an implanted prosthetic valve from a location within a patient's body. The implanted prosthetic valve may include a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level. A plurality of prosthetic valve leaflets may be positioned within the flow channel and extend inward from the inner surface of the valve body.

In embodiments herein, an implantable prosthetic valve is disclosed, including a plurality of prosthetic valve leaflets and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

In embodiments herein, an implantable prosthetic valve is disclosed, including a valve body having a proximal end, a distal end, an outer surface, and an inner surface facing a flow channel, and a first skirt forming at least a portion of the outer surface of the valve body and configured to be bioresorbable, and a second skirt positioned radially inward of the first skirt and configured to be thromboresistant. A plurality of prosthetic valve leaflets may be positioned within the flow channel and extend inward from the inner surface of the valve body.

In embodiments herein, a method is disclosed, including implanting a prosthetic valve within a patient's body. The prosthetic valve may include a plurality of prosthetic valve leaflets and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

In embodiments herein, a method is disclosed, including removing an implanted prosthetic valve from a location within a patient's body. The implanted prosthetic valve upon implantation may include a plurality of prosthetic valve leaflets, and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

In embodiments herein, an implantable prosthetic valve is disclosed, including a valve body having an outer surface and an inner surface facing a flow channel, and a skirt including an exterior surface forming at least a portion of the outer surface of the valve body and configured to be porous to allow tissue ingrowth within the exterior surface and an interior surface that is configured to be thromboresistant. A plurality of prosthetic valve leaflets may be positioned within the flow channel and extending inward from the inner surface of the valve body.

In embodiments herein, a method is disclosed, including implanting a prosthetic valve within a patient's body. The prosthetic valve may include a valve body having an outer surface and an inner surface facing a flow channel, and a skirt including an exterior surface forming at least a portion of the outer surface of the valve body and configured to be porous to allow tissue ingrowth within the exterior surface and an interior surface that is configured to be thromboresistant. A plurality of prosthetic valve leaflets may be positioned within the flow channel and extending inward from the inner surface of the valve body.

In embodiments herein, an implantable prosthetic valve is disclosed, including a valve body having an outer surface and an inner surface facing a flow channel, and a skirt including porous expanded polytetrafluoroethylene (ePTFE) to allow tissue ingrowth within the skirt. A plurality of prosthetic valve leaflets may be positioned within the flow channel and extending inward from the inner surface of the valve body.

In embodiments herein, a method is disclosed, including implanting a prosthetic valve within a patient's body. The prosthetic valve may include a valve body having an outer surface and an inner surface facing a flow channel, and a skirt including porous expanded polytetrafluoroethylene (ePTFE) to allow tissue ingrowth within the skirt. A plurality of prosthetic valve leaflets may be positioned within the flow channel and extending inward from the inner surface of the valve body.

### BRIEF DESCRIPTION OF THE DRAWINGS

Features and advantages of the systems, devices, and methods as disclosed herein will become appreciated as the same become better understood with reference to the specification, claims, and appended drawings wherein:

FIG. 1 illustrates a perspective view of an implantable prosthetic valve according to embodiments herein.

FIG. 2 illustrates a top view of the implantable prosthetic valve shown in FIG. 1.

FIG. 3 illustrates a top view of the implantable prosthetic valve shown in FIG. 1, with prosthetic valve leaflets moved from the position shown in FIG. 2.

FIG. 4 illustrates a cross sectional schematic view of the implantable prosthetic valve shown in FIG. 1.

FIG. 5 illustrates a schematic view of a skirt of a prosthetic valve according to embodiments herein.

FIG. 6 illustrates a side view of a deployment apparatus according to embodiments herein.

FIG. 7 illustrates a schematic view of the implantable prosthetic valve shown in FIG. 1 being deployed to a native aortic valve.

FIG. 8 illustrates a schematic view of the implantable prosthetic valve shown in FIG. 1 implanted at a native aortic valve.

FIG. 9 illustrates a schematic view of the implantable prosthetic valve shown in FIG. 1 implanted at a native aortic valve.

FIG. 10 illustrates a schematic view of the implantable prosthetic valve shown in FIG. 1 implanted at a native aortic valve.

FIG. 11 illustrates a schematic view of the implantable prosthetic valve shown in FIG. 1, with the leaflets of a native aortic valve severed.

FIG. 12 illustrates a schematic view of the implantable prosthetic valve shown in FIG. 1 being removed from a native aortic valve.

FIG. 13 illustrates a cross sectional schematic view of an implantable prosthetic valve according to embodiments herein.

FIG. 14 illustrates a schematic view of the implantable prosthetic valve shown in FIG. 13 implanted at a native aortic valve.

FIG. 15 illustrates a schematic view of the implantable prosthetic valve shown in FIG. 13 being removed from a native aortic valve.

FIG. 16 illustrates a cross sectional schematic view of an implantable prosthetic valve according to embodiments herein.

FIG. 17 illustrates a schematic view of the implantable prosthetic valve shown in FIG. 16 implanted at a native aortic valve.

FIG. 18 illustrates a schematic view of the implantable prosthetic valve shown in FIG. 16 implanted at a native aortic valve.

FIG. 19 illustrates a schematic view of the implantable prosthetic valve shown in FIG. 16 being removed from a native aortic valve.

FIG. 20A illustrates a perspective view of an implantable prosthetic valve according to embodiments herein.

FIG. 20B illustrates a cross sectional schematic view of the implantable prosthetic valve shown in FIG. 20A.

FIG. 21A illustrates a cross sectional view of a skirt of an implantable prosthetic valve.

FIG. 21B illustrates a cross sectional view of a skirt of an implantable prosthetic valve.

FIG. 21C illustrates a cross sectional view of a skirt of an implantable prosthetic valve.

FIG. 21D illustrates a cross sectional view of a skirt of an implantable prosthetic valve.

FIG. 22 illustrates a front schematic view of skirt layers of an implantable prosthetic valve.

FIG. 23A illustrates a partial isolated perspective view of a skirt of an implantable prosthetic valve.

FIG. 23B illustrates a partial isolated perspective view of a skirt of an implantable prosthetic valve.

FIG. 24 illustrates a schematic view of the implantable prosthetic valve shown in FIG. 20A implanted at a native aortic valve.

FIG. 25 illustrates a schematic view of the implantable prosthetic valve shown in FIG. 20A implanted at a native aortic valve.

FIG. 26 illustrates a schematic view of the implantable prosthetic valve shown in FIG. 20A implanted at a native aortic valve.

### DETAILED DESCRIPTION

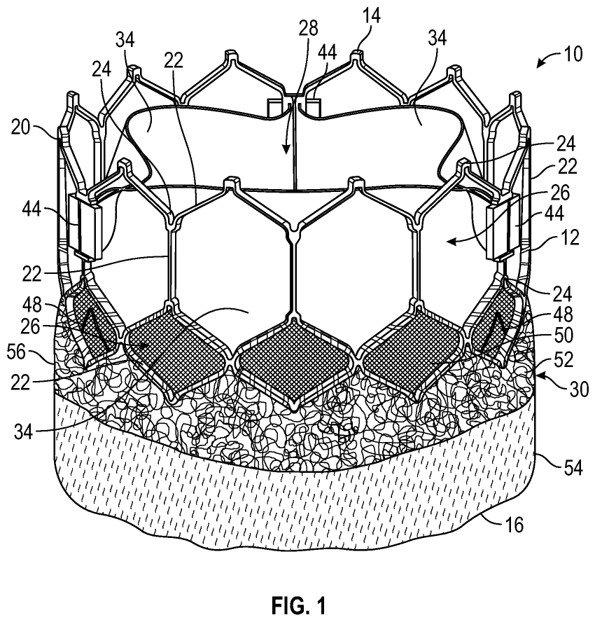

FIG. 1 illustrates a perspective view of an implant in the form of an implantable prosthetic valve 10. The prosthetic valve 10 may be configured to be deployed within a portion of a patient's body. The prosthetic valve 10, for example, may be deployed within a native heart valve annulus, which may comprise a native aortic valve, or in embodiments may comprise a native mitral, tricuspid, or pulmonary valve.

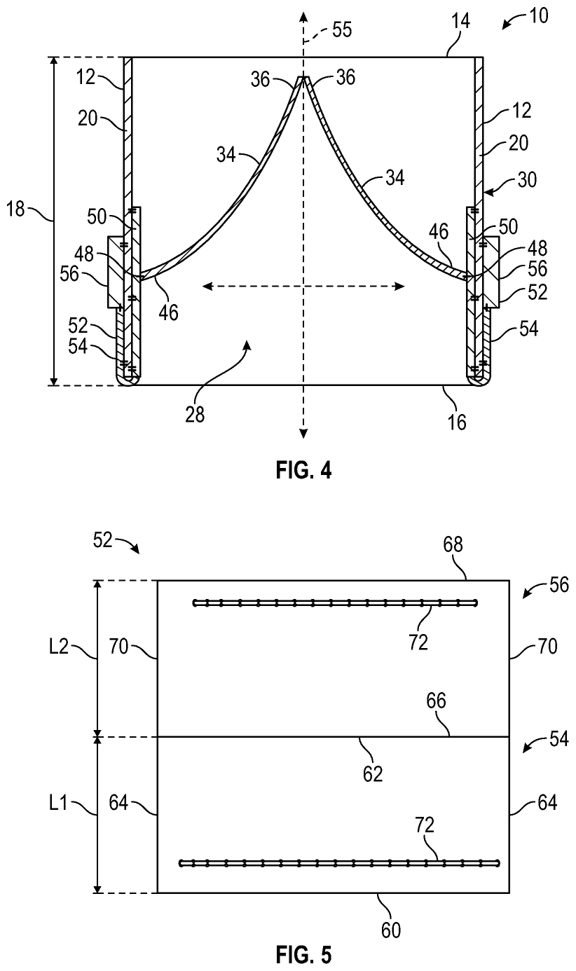

The prosthetic valve 10 may include a valve body 12 that may have a proximal end 14, a distal end 16, and a length 18 (as marked in FIG. 4) between the proximal end 14 and the distal end 16. The proximal end 14 may comprise an outflow end of the prosthetic valve 10, and the distal end 16 may comprise an inflow end of the prosthetic valve 10.

The valve body 12 may have a cylindrical shape as shown in FIG. 1, or may have another shape (e.g., a tapered or “V” shape, or a bulb shape, or other desired shape).

The valve body 12 may include a frame 20 that may include a plurality of struts 22 that may join together at junctures 24 and may have spaces 26 between the struts 22. The spaces 26 may comprise openings of the frame 20 that may allow fluid flow therethrough or the passage of other components therethrough. The spaces 26 may be configured to reduce the overall weight of the frame 20, and also allow the frame 20 to be compressed to reduce a diameter of the frame 20 and be expanded to increase a diameter of the frame 20. The configuration of the frame may vary in other embodiments.

The frame 20 may be configured to allow the valve body 12 to be collapsible and expandable, with the frame 20 being compressed (or crimped) to move to a collapsed (or undeployed or unexpanded) state and being expanded to move to an expanded (or deployed) state. The frame 20 may be configured to be radially compressed and axially lengthened while being radially compressed.

The struts 22 may be configured such that as the frame 20 is compressed to reduce a diameter of the frame 20, the length of the frame 20 may increase. Also, as the frame 20 is expanded to increase the diameter of the frame 20, the length of the frame 20 may decrease. The frame 20 may be compressed in a variety of manners, including use of a crimping device, and may be expanded in a variety of manners, including being expanded with a balloon, being self-expandable, or being mechanically expandable. Embodiments herein may be shown as a balloon expandable implant, yet self-expandable implants or mechanically expandable implants may be utilized as well.

The plurality of struts 22 may be configured to move closer together to allow the frame 20 to move to the collapsed state. The width of the spaces 26 between the struts 22 may be reduced as the frame 20 is moved to the collapsed (or undeployed or unexpanded) state, with the length of the spaces 26 increasing. The struts 22 of the frame 20 may be configured to circumferentially move away from each other to move to the expanded state. The width of the spaces 26 between the struts 22 may be increased as the frame 20 is moved to the expanded (or deployed) state, with the length of the spaces 26 decreasing.

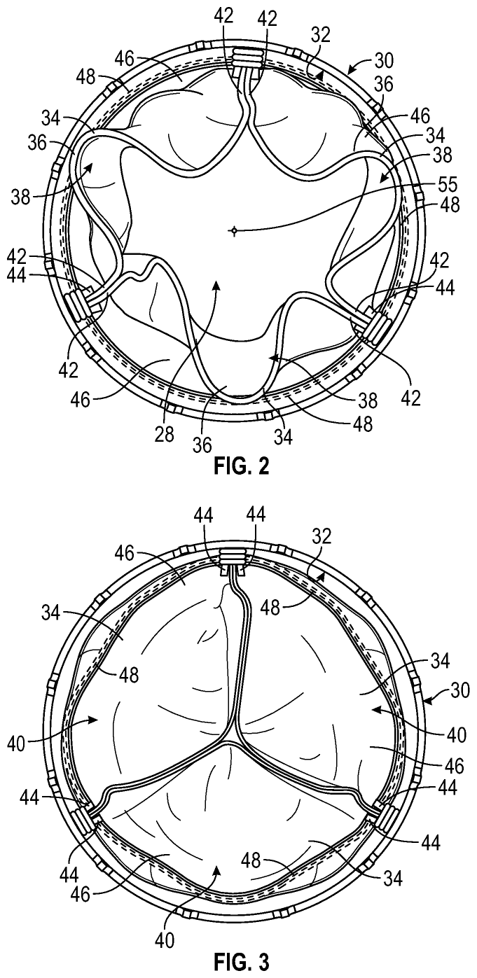

The valve body 12 may surround a flow channel 28 that may allow for flow of fluid (e.g., blood or another fluid) through the valve body 12. The valve body 12 may include an outer surface 30 that may face outward from the valve body 12 and may include an inner surface 32 (as marked in FIGS. 2 and 3) that faces opposite the outer surface 30 and faces the flow channel 28. The outer surface 30 may comprise an anchoring surface that may be utilized to anchor the prosthetic valve 10 within the desired portion of the patient's body (e.g., a heart valve annulus if desired). The outer surface 30 may apply a force radially outward to anchor the prosthetic valve 10 within an annulus.

A plurality of prosthetic valve leaflets 34 may be positioned within the flow channel 28 and may extend inward from the inner surface 32 of the valve body 12. The plurality of valve leaflets 34 may be configured to move away from each other to move to an open position (as shown in FIG. 2) and may be configured to move towards each other to move to a closed position (as shown in FIG. 3). The plurality of valve leaflets 34 may each include an upper end portion 36 (marked in FIG. 2), and the upper end portions 36 of the plurality of valve leaflets 34 are configured to contact each other to close the flow channel 28 of the prosthetic valve 10 when the leaflets 34 are in the closed position. The upper end portions 36 are configured to move away from each other to open the flow channel 28 of the implantable prosthetic valve 10 when the plurality of valve leaflets 34 are in the open position (as shown in FIG. 2). The plurality of valve leaflets 34 may move back and forth between open and closed positions or states or configurations to replicate the motion of a native valve.

Each valve leaflet 34 may include an interior surface 38 (marked in FIG. 2) configured to face towards the flow channel 28 of the implantable prosthetic valve 10. Each valve leaflet 34 may include an exterior surface 40 (marked in FIG. 3) facing opposite the interior surface 38 and facing away from the flow channel 28 of the prosthetic valve 10. Portions of the interior surface 38 of respective leaflets 34 may contact each other when the leaflets 34 move to the closed position.

Each valve leaflet 34 may include outer end portions 42 (marked in FIG. 2) that couple to the frame 20 of the valve body 12. The outer end portions 42 may couple to the frame 20 at commissure points of the leaflets 34 and may pass through openings of the frame 20 to couple to the frame 20. The coupling may have a variety of forms. For example, each valve leaflet 34 may include tabs 44 at the respective outer end portion 42 of the valve leaflet 34. The tabs 44 may extend through openings in the frame 20 to couple to the frame 20 and then may be sutured to hold the tabs 44 in position.

Further, each valve leaflet 34 may include a lower end portion 46 that may be sutured to a skirt 50 along a scallop line 48 that may comprise a suture line. For example, a lower end portion 46 of each leaflet 34 opposite the upper end portion 36 may be sutured to the skirt 50 (marked in FIGS. 1 and 4) at a scallop line 48. The sutures of the scallop line 48 may hold the leaflets 34 to the frame 20 and prevent undesired fluid flow through the prosthetic valve 10 outside of the flow channel 28.

The valve leaflets 34 may be configured to open and close during operation such that the proximal end 14 of the prosthetic valve 10 forms an outflow end of the prosthetic valve 10, and the distal end 16 of the prosthetic valve 10 forms an inflow end of the prosthetic valve 10. The valve leaflets 34 may be configured to impede fluid flow in an opposite direction from the outflow end to the inflow end of the prosthetic valve 10 when the valve leaflets 34 are in a closed position.

Referring to FIG. 1, the valve body 12 may include one or more skirts 50, 52. The skirt 50 may comprise a skirt that is positioned inward of the frame 20 and may be coupled to the plurality of valve leaflets 34 via sutures or another form of coupler. For example, the scallop line 48 may be present on the skirt 50. The skirt 50 may form at least a portion of the inner surface 32 of the valve body 12 and may extend around the entire interior perimeter of the frame 20. The skirt 50 in embodiments may be coupled to the frame 20 via sutures or another form of coupler.

The skirt 52 may comprise an outer skirt that may form at least a portion of the outer surface 30 of the valve body 12. The skirt 52 may be positioned radially outward of the plurality of prosthetic valve leaflets 34, radially outward of the skirt 50 (which may be considered an inner skirt), and may be positioned radially outward of the frame 20 in embodiments.

The skirt 52 may extend circumferentially about the valve body 12, and may extend circumferentially around the entirety of the valve body 12. The skirt 52 may cover an outer surface of the frame 20 at a distal portion of the frame 20, and may extend for only a portion of the axial length of the frame 20 (e.g., a distal portion), or may extend for the entire axial length of the frame 20. The skirt 52 may be coupled to the frame 20 via sutures or another form of coupler, which may thus couple the skirt 52 to the other components of the valve 10 (including the valve leaflets 34 and the skirt 50).

FIG. 2 illustrates a top view of the implantable prosthetic valve 10 with the valve leaflets 34 in an opened configuration. FIG. 3 illustrates a top view of the implantable prosthetic valve 10 with the valve leaflets 34 in a closed configuration.

FIG. 4 illustrates a schematic cross sectional view of the implantable prosthetic valve 10. Certain features of the implantable prosthetic valve 10 have been excluded for purposes of clarity. Referring to FIGS. 1 and 4, and with reference to the skirt 52, the skirt 52 may include at least two levels, with each level forming at least a portion of the outer surface 30 of the valve body 12.

The skirt 52, for example, may include a first level 54 that forms at least a portion of the outer surface 30 of the valve body 12 and is configured to be thromboresistant. The first level 54 may be positioned adjacent to the distal end 16 of the valve body 12. The first level 54, in embodiments, may have a distal end that wraps over the distal end of the frame 20 and may couple to the interior skirt 50. The first level 54 may extend proximally from the distal end 16 of the implantable prosthetic valve 10 to a proximal end of the first level 54. The first level 54 may have a length (L1, marked in FIG. 5) along an axis 55 that the implantable prosthetic valve 10 extends around.

The first level 54 may extend circumferentially about the valve body 12 and may extend circumferentially about the entirety of the valve body 12. The first level 54 may form a band that extends around the valve body 12. The first level 54 may be positioned radially outward of the frame 20.

Referring to FIGS. 1 and 4, the skirt 52 may include a second level 56 that forms at least a portion of the outer surface 30 of the valve body 12 and is configured to allow tissue ingrowth with the second level 56. The second level 56 may be positioned between the first level 54 and the proximal end 14 of the valve body 12. The second level 56 may be positioned adjacent to the first level 54 in an axial direction (for example, along the axis 55). The second level 56 may be positioned proximal of the first level 54 and the first level 54 may be positioned distal of the second level 56. The second level 56 may extend proximally from a distal end of the second level 56 to a proximal end of the second level 56. The second level 56 may have a length (L2, marked in FIG. 5) along an axis 55 that the implantable prosthetic valve 10 extends around.

The second level 56 may extend circumferentially about the valve body 12 and may extend circumferentially about the entirety of the valve body 12. The second level 56 may form a band that extends around the valve body 12. The second level 56 may be positioned radially outward of the frame 20.

The second level 56 may be coupled to the first level 54 via bonding, stitching, or another form of coupling. For example, the distal end of the second level 56 may be coupled to the proximal end of the first level 54. In embodiments, the first level 54 and second level 56 may both be coupled to the frame 20 by bonding, stitching, or another form of coupling.

FIG. 5 illustrates a schematic view of the outer skirt 52, showing the outer skirt 52 decoupled from the frame 20 and unrolled and laid flat in plane with the page. The first level 54 may include a distal end 60, and a proximal end 62 spaced axially from the distal end 60. The first level 54 may further comprise axially extending edges 64 that may join with each other when placed upon the frame 20 in embodiments. The first level 54 may have an axial length L1 between the distal end 60 and the proximal end 62.

The second level 56 may include a distal end 66, and a proximal end 68 spaced axially from the distal end 66. The second level 56, may further comprise axially extending edges 70 that may join with each other when placed upon the frame 20 in embodiments. The second level 56 may have an axial length L2 between the distal end 66 and the proximal end 68.

In embodiments, the distal end 66 of the second level 56 may be coupled to the proximal end 62 of the first level 54 via bonding, stitching, or another form of coupling. In various embodiments, the distal end 66 of the second level 56 may overlap the proximal end 62 of the first level 54 to define an overlapping portion therebetween. In embodiments, the first level 54 and second level 56 may both be coupled to the frame 20 by suture lines 72 or another form of coupling.

The lengths L1, L2 of the first level 54 and the second level 56 may be set as desired based on a desired implementation of the implantable prosthetic valve 10. In embodiments, a ratio of the relative axial extent along the valve body of the first level 54 to the second level 56 may be 1:1 or greater. For example, L1 may represent at least 50% of the total length of the outer skirt 52, with L2 representing 50% or less. In embodiments, L1 may represent at least 60%, with L2 representing 40% or less. In embodiments, L1 may represent at least 70%, with L2 representing 30% or less. Greater or lesser proportions may be utilized as desired. Various ratios, may be utilized, for example at least 3:2, or 7:3, as a ratio of L1 to L2. In embodiments, L2 may represent a greater proportion of the total length of the outer skirt 52 than L1 (e.g., a ratio of 1:2 or lesser, or 2:3 or lesser).

As discussed, the first level 54 may be configured to be thromboresistant, and the second level 56 may be configured to allow tissue ingrowth with the second level 56. The respective materials of the first level 54 and the second level 56 may be different to produce such a result. For example, the first level 54 may include a material that is configured to be thromboresistant and the second level 56 may include a material that is configured to allow tissue ingrowth with the second level 56.

The first level 54, for example, may include at least one or more of a polytetrafluoroethylene (PTFE), an ultra high molecular weight polyethlene (UHMWPE), or a coated thermoplastic polyurethane (TPU). The polytetrafluoroethylene (PTFE) may comprise expanded polytetrafluoroethylene (ePTFE) in embodiments as desired. Various other thromboresistant materials may be utilized as desired. In embodiments, a thromboresistant coating may be utilized with the first level 54. For example, a coating of PTFE or ePTFE may be applied to material of the first level 54, which may comprise polyethylene terephthalate (PET) or another form of material. The underlying material of the first level 54 in such an embodiment, for example, may be a material that allows for tissue ingrowth, such as PET, yet is coated with a thromboresistant coating. In embodiments, the material of the first level 54 may be fabricated to be thromboresistant. For example, a knit pattern of the material of the first level 54 may be configured to be thromboresistant, with micropatterns or other forms of knit patterns. A knit PTFE fabric may be utilized in embodiments. The material of the first level 54 may include a smooth texture to be thromboresistant and inhibit tissue growth. Combinations of such features may be utilized to result in a thromboresistant first level 54.

The second level 56, for example, may include polyethylene terephthalate (PET) or another form of material that is configured to allow tissue ingrowth. In embodiments, a coating may be utilized with the second level 56 that is configured to allow tissue ingrowth. For example, a porous coating or other form of coating may be applied to a thromboresistant material such as ultra high molecular weight polyethlene (UHMWPE) to allow for tissue ingrowth. The underlying material of the second level 56 in such an embodiment, for example, may be a material that inhibits tissue ingrowth, yet is coated with a coating that allows tissue ingrowth. For example, in embodiments, the underlying material of the first level 54 and second level 56 may be the same, with one or more coatings applied to either the first level 54 or second level 56 or both to produce the different properties of the levels 54, 56 disclosed herein. In embodiments, the material of the second level 56 may be fabricated to allow tissue ingrowth. For example, a knit pattern of the material of the second level 56 may be configured to allow tissue ingrowth, with a large knit pattern or other forms of knit patterns. A knit PET fabric may be utilized in embodiments. In embodiments, the second level 56 may include textured yarns extending radially outward. The material of the second level 56 may include a porous texture to allow for tissue ingrowth. Combinations of such features may be utilized to result in a second level 56 that allows for tissue ingrowth.

Various other materials for the first level 54 and the second level 56 may be utilized as desired.

The first level 54 may be configured to be thromboresistant to reduce the possibility of tissue adhesion with the portion of the patient's body to which the prosthetic valve 10 is implanted. Such a result may be desired in the possibility of removing the prosthetic valve 10 from the implantation site, in which a lack of tissue adhesion may allow for an easier removal process. For example, a reduced possibility of cutting of tissue may be required, which may reduce trauma to the patient's body upon removal of the prosthetic valve 10.

The second level 56 may be utilized to allow for tissue ingrowth however, to allow for some tissue adhesion to the native implantation site. Such tissue adhesion may enhance the anchoring of the prosthetic valve 10 to the native implantation site and may enhance sealing of fluid flow around the prosthetic valve 10, which may comprise paravalvular sealing.

The first level 54 may be positioned such that the portion of the native anatomy surrounding the first level 54 may have a reduced possibility of adhering to the first level 54 due to the thromboresistant properties of the first level 54. In an embodiment in which the prosthetic valve 10 is implanted to a native heart valve, the first level 54 may be positioned within the annulus and surrounded by the annulus of the native heart valve.

The second level 56 may be positioned such that the portion of the native anatomy surrounding the second level 56 may be severed and removed during removal of the prosthetic valve 10. In an embodiment in which the prosthetic valve 10 is implanted to a native heart valve, the second level 56 may be surrounded by the leaflets of the native heart valve. The second level 56 may be configured to allow tissue ingrowth with native leaflets of the native heart valve. Thus, during a removal procedure, the native leaflets may be severed and removed from the patient's body with the prosthetic valve As such, during a removal procedure, only the native leaflets may need to be severed, and not a portion of the annulus, which may reduce the possibility of unwanted tissue damage and trauma during the removal procedure.

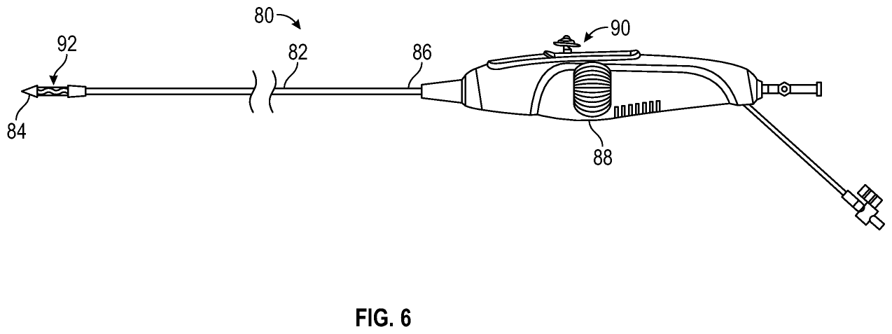

FIG. 6 illustrates a deployment apparatus 80 that may be utilized in embodiments herein. The deployment apparatus 80 may include an elongate shaft 82 having a distal end 84 and a proximal end 86. The proximal end 86 of the elongate shaft 82 may couple to a handle 88 that may be gripped and may be utilized to control the movement of the elongate shaft 82. The handle 88 for example, may include a deflection mechanism 90 that may be configured to be operated to control deflection of the elongate shaft 82 to navigate the elongate shaft 82 to the desired location.

The distal end 84 of the elongate shaft 82 may include an implant retention area 92 that may be configured to retain an implant, such as the implantable prosthetic valve 10 therein. The implant retention area 92, in embodiments, may include an inflation balloon or other deployment mechanism that may be utilized to deploy the implant. For example, a capsule may be retracted to allow the implant to self-expand, or a mechanism may be utilized to mechanically deploy the implant. In embodiments, various forms of deployment apparatuses may be utilized as desired.

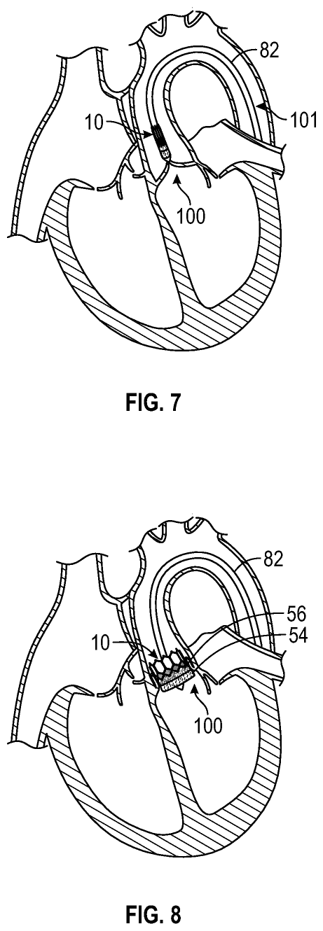

FIGS. 7-9 illustrate an exemplary method of implantation of an implant such as the implantable prosthetic valve 10 within a patient's body. The steps of the method may be varied as desired, which may include adding, removing, or modifying features, or substituting features across embodiments. FIG. 7 illustrates the implantable prosthetic valve 10 coupled to the elongate shaft 82 of the deployment apparatus 80 shown in FIG. 6 for example.

The implantable prosthetic valve 10 may be advanced transcatheter and transvascular to the desired implantation site, which is a native heart valve such as the native aortic valve 100 as shown in FIG. 7. In embodiments, other forms of implantation sites may be utilized, for example different heart valves (e.g., mitral, pulmonary, or tricuspid valves), or other implantation sites within the patient's body.

The elongate shaft 82 may deflect around the aortic arch 101, for example, to approach the native aortic valve 100, or other approaches may be utilized as desired. Transapical or other surgical approaches may be utilized as desired.

The prosthetic valve 10 may be deployed to a desired treatment site within a patient's body. The treatment site may be an implantation site for the prosthetic valve 10 to be implanted within the patient's body. The treatment site may be a valve of the patient's body, which may be a native valve, or may comprise a previously deployed prosthetic valve within the patient's body. The treatment site may include leaflets, which may be native leaflets (of a native valve) or may be host leaflets (of a previously deployed valve or host valve). The leaflets may suffer from a variety of maladies that may require the leaflets to be replaced with the leaflets of the prosthetic valve 10. For example, calcification of the leaflets or other conditions of the valve (e.g., stenosis or other conditions) may require the operation of the leaflets to be replaced with the leaflets of the prosthetic valve 10. The leaflets may remain within the patient's body upon the prosthetic valve 10 being deployed. The prosthetic valve 10 may be deployed in between the native or host leaflets such that the native or host leaflets are pushed radially outward upon expansion of the prosthetic valve. Such a configuration may result whether the prosthetic valve 10 is deployed within a native valve, or within a host valve that has been previously deployed.

In embodiments, the prosthetic valve 10 may have a variety of forms, including a balloon expandable valve or a mechanically expandable valve as desired. Self-expanding valves may also be utilized. The delivery system utilized to deploy the valve 10 may be configured to expand the valve 10 according to the desired method of expansion (balloon expandable, mechanically expandable, self-expandable, among others).

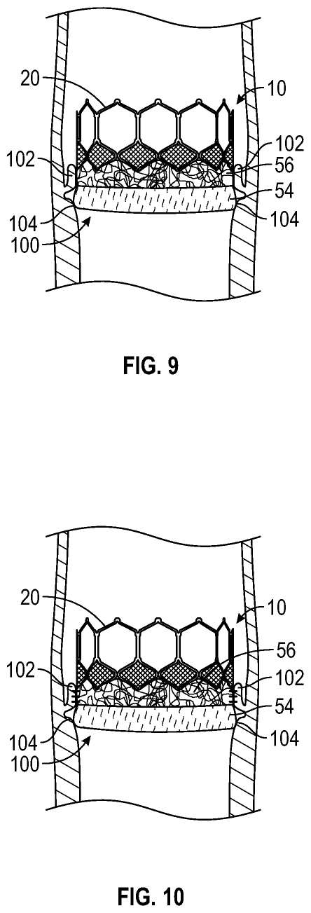

FIG. 8, for example, illustrates the prosthetic valve 10 having been expanded and implanted to the native aortic valve 100. In embodiments, the force of the outer surface 30 of the valve body 12 against the native valve leaflets and the native valve annulus may anchor the prosthetic valve 10 to the native aortic valve 100. Notably, the second level 56 may be positioned such that the second level 56 contacts the native valve leaflets, and preferably not the native valve annulus. FIG. 9, for example, represents a view of the prosthetic valve 10 having been implanted to the native aortic valve 100.

Referring to FIG. 9, the prosthetic valve 10 is shown implanted to the native aortic valve 100. The second level 56 is shown to contact one or more native valve leaflets 102, and preferably not the native valve annulus 104. The first level 54, however, is shown to be positioned at the native valve annulus 104 and in contact with the native valve annulus 104. As discussed herein, the first level 54 may be configured to be thromboresistant, thus reducing the possibility of tissue adhesion with the native valve annulus 104. The second level 56 may allow for tissue ingrowth, which may occur with the native valve leaflets 102. Such tissue ingrowth may comprise neointimal tissue.

FIG. 10, for example, illustrates the prosthetic valve 10 having been implanted for a duration, which has resulted in tissue adhesion between the second level 56 and the native valve leaflets 102. The first level 54, however, has not adhered to the native valve annulus 104, which may enhance the ease with which the prosthetic valve 10 may be removed (in an explant procedure). For example, if the prosthetic valve 10 should be desired to be removed for a reason, then preferably the lack of tissue adhesion around the first level 54 may reduce the need for cutting tissue at the native valve annulus 104. The second level 56 may include tissue adhesion, which may improve anchoring and sealing of the prosthetic valve 10. However, the tissue adhesion may be with the native valve leaflets 102, which may only be required to be severed in an explant procedure.

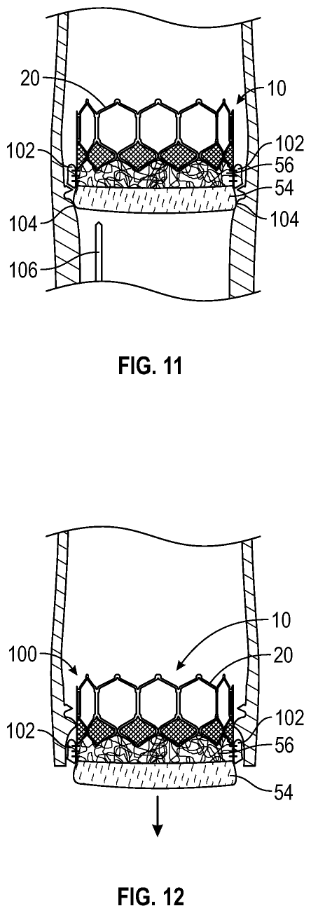

FIG. 11, for example, illustrates a step in a removal or explant procedure. The implanted prosthetic valve may be removed from the location in the patient's body, such as the native heart valve. The removal or explant procedure may comprise a surgical procedure or a minimally invasive procedure, which may transcatheter if desired. A cutting device 106 (e.g., a scalpel or other form of blade or laser or other form of cutting device) may sever the leaflets 102 that are adhered to the second level 56. The native leaflet 102 may be severed from the patient's body while being in contact with the second level 56. The native leaflet 102 may be bonded to the second level 56. The cutting device 106 notably has not cut any tissue at the native valve annulus 104 due to the lack of tissue adhesion at this portion. FIG. 12 illustrates the prosthetic valve 10 being removed from the native aortic valve 100 with the native valve leaflets 102. The method may include removing the native leaflet 102 and the second level 56 from the patient's body along with the remainder of the prosthetic valve 10. The area that the native aortic valve 100 occupied may be available for implantation of another prosthetic heart valve.

Variations in the configuration and use of the prosthetic valve 10 may be provided. Variations in the steps of the methods disclosed herein may be provided.

In embodiments, the ratio of the length of the first level 54 relative to the second level 56 may be set such that the second level 56 does not contact the native valve annulus 104 when implanted. In embodiments, the length of the second level 56 may be configured such that no portion of the second level 56 contacts the annulus. In embodiments, more than two levels of skirt may be provided (e.g., a multi-level skirt having at least two levels, or two or more levels). The multiple levels of skirt may include one or more levels configured with similar materials as the first level 54 and one or more levels configured with similar materials as the second level 56. In embodiments, a thromboresistant level may comprise the distalmost level of a prosthetic valve, to reduce the possibility of adhesion with the annulus. Various other configurations and uses of the prosthetic valve 10 may be provided.

The features of the prosthetic valve 10 may be implemented independently or in combination with other features disclosed herein.

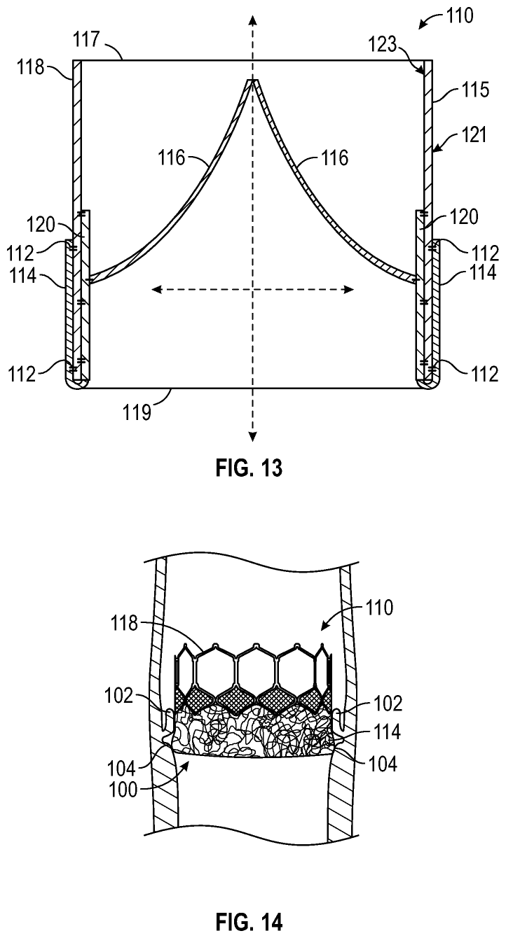

FIG. 13 illustrates a schematic cross sectional view of an embodiment of an implantable prosthetic valve 110 including one or more bioresorbable couplers 112 that may be utilized to couple a skirt 114 to the remainder of the implantable prosthetic valve 110, including the plurality of prosthetic valve leaflets 116.

The skirt 114 may comprise a body that forms at least a portion of the outer surface of the valve body 115 and may be utilized to anchor the valve body 115 at the implantation site and promote sealing at the implantation site. The skirt 114 may extend circumferentially about the valve body 115 and may extend around the entirety of the valve body 115. The skirt 114 may be constructed of materials that are configured to allow tissue ingrowth with the skirt 114, as discussed in regard to the second level 56 of the skirt 52 discussed in regard to FIGS. 1-12, or may have another configuration as desired. For example, the skirt 114 may be made of a knit PET fabric, among other forms of materials.

The valve body 115 may further include a frame 118 that may be configured similarly as the frame 20 shown in FIG. 1, or may have another configuration. The valve body 115, similar to the valve body 12, may include a proximal end 117, a distal end 119, an outer surface 121, and an inner surface 123 facing a flow channel in which the plurality of prosthetic valve leaflets 116 are positioned.

The valve body 115 may further include a skirt 120 that may be positioned radially inward of the frame 118, and may be configured similarly as the skirt 50 shown in FIGS. 1 and 4. The plurality of prosthetic valve leaflets 116 may be positioned radially inward of the skirt 120.

The skirt 114 (which may be referred to as an outer skirt), may couple directly to the frame 118 via one or more bioresorbable couplers 112, or may couple to the skirt 120 (which may be referred to as an inner skirt) via one or more bioresorbable couplers 112, or a combination of the frame 118 and the skirt 120. The skirt 114 in embodiments may be positioned radially outward of the frame 118. The coupling of the skirt 114 to the frame or inner skirt 120 may couple the skirt 114 to the prosthetic valve leaflets 116. The skirt 114 in embodiments may couple directly to the plurality of prosthetic valve leaflets 116 with the bioresorbable couplers 112. The skirt 114 in embodiments, may include one or more bioresorbable couplers 112 that may couple to a scallop line or suture line of the plurality of prosthetic valve leaflets 116 as desired. One or more of the bioresorbable couplers 112 may couple to the scallop line or suture line, and other bioresorbable couplers 112 may couple to the frame 118 or the skirt 120 as desired. Other locations of coupling, or combinations of locations of coupling may be utilized as desired.

The bioresorbable couplers 112 may comprise bioresorbable sutures in embodiments, or other forms of couplers as desired (e.g., tabs, hooks, pins, adhesive bodies, or other forms of couplers). The bioresorbable couplers 112 may be configured to dissolve while positioned within the patient's body, thus reducing the coupling between the skirt 114 and the remainder of the valve body 115. The bioresorbable coupler 112 may be configured to dissolve in a resorb process. As such, during a removal or explant procedure, the skirt 114 may be more easily separated from the prosthetic valve leaflets 116 or frame 118, to allow such components to be removed from the implantation site while the skirt 114 may remain in place if desired. The skirt 114 may be configured to separate from the plurality of prosthetic valve leaflets 116 upon dissolving of the one or more bioresorbable couplers 112. For example, the skirt 114 may be configured to configured to allow tissue ingrowth with the skirt 114. If the skirt 114 has adhered to the implantation site via tissue adhesion, then the skirt 114 may be separated more easily during a removal procedure if the bioresorbable couplers 112 have dissolved.

The inner skirt 120, however, in embodiments may be configured to be thromboresistant, and may be configured of similar materials discussed in regard to the first level 54 of the skirt 52 discussed in regard to FIGS. 1-12. For example, the inner skirt 120 may comprise a PTFE or ePTFE material (among other forms of material). The inner skirt 120 further may couple to the frame 118 and the prosthetic valve leaflets 116 with non-bioresorbable couplers (such as non-resorbable sutures or another form of coupler). Thus, the inner skirt 120 may not bond via tissue adhesion and the non-bioresorbable couplers may remain secure following implantation.

FIG. 14, for example, illustrates the implantable prosthetic valve 110 having been implanted within a patient's body to a native heart valve such as a native aortic valve 100. The skirt 114 may be contacted to the native valve leaflets 102 and the native valve annulus 104 if desired. Further, tissue adhesion may occur between the skirt 114 and such features of the native aortic valve 100 in embodiments. The bioresorbable couplers 112 shown in FIG. 13 may dissolve over time, thus reducing the coupling between the skirt 114 and the remainder of the implantable prosthetic valve 110. The skirt 114 may be configured to separate from the plurality of prosthetic valve leaflets 116 upon the dissolving of the one or more bioresorbable couplers 112.

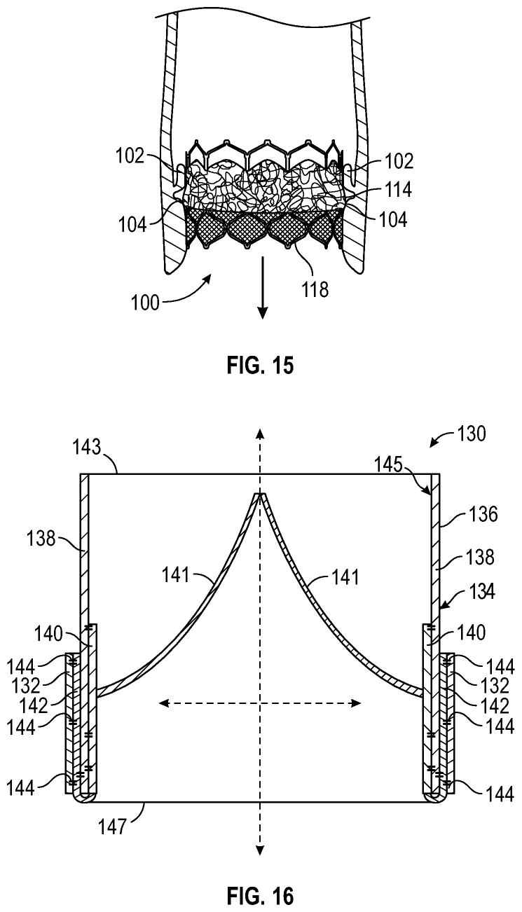

FIG. 15 illustrates a process in a removal or explant procedure, in which the implanted prosthetic valve is removed from a location within the patient's body. The implanted prosthetic valve upon implantation may be configured as shown in FIGS. 13 and 14. The skirt 114 may be separated from the frame 118 and the prosthetic valve leaflets 116 (marked in FIG. 13) in such a procedure while removing the implanted prosthetic valve 110 from the native heart valve. The one or more bioresorbable couplers 112 have dissolved. The separation of the skirt 114 from the frame 118 may enhance the ease of the removal or explant procedure, and may reduce the need for cutting of native tissue during a removal or explant procedure. The prosthetic valve 110 may be removed from the patient's body without the skirt 114.

The features of the prosthetic valve 110 may be implemented independently or in combination with other features disclosed herein.

FIG. 16 illustrates a schematic cross sectional view of an embodiment of an implantable prosthetic valve 130 including a skirt 132 that is configured to be bioresorbable. The bioresorbable skirt 132 in embodiments may comprise at least a portion of the outer surface 134 of the valve body 136. The bioresorbable skirt 132 may be constructed of a bioresorbable fabric or other bioresorbable materials.

The valve body 136 may include a frame 138 and may include a skirt 140 that may be positioned interior of the frame 138 if desired. The skirt 140 may be configured similarly as the skirt 50 shown in FIGS. 1 and 4. The plurality of prosthetic valve leaflets 141 may be positioned radially inward of the skirt 140. The valve body 136, similar to the valve body 12, may include a proximal end 143, a distal end 147, an outer surface 134, and an inner surface 145 facing a flow channel in which the plurality of prosthetic valve leaflets 141 are positioned.

The bioresorbable skirt 132 may be positioned radially outward of the frame 138 in embodiments and radially outward of the skirt 140. The bioresorbable skirt 132 (which may be referred to as an outer skirt) may extend circumferentially about the frame 138 and may extend circumferentially about the entirety of the frame 138.

In embodiments, a skirt 142 (which may be referred to as a main skirt) may be positioned radially inward of the bioresorbable skirt 132. In embodiments, the skirt 142 may be positioned radially outward of the frame 138 and radially inward of the bioresorbable skirt 132. The skirt 142 accordingly may be positioned between the frame 138 and the bioresorbable skirt 132 in embodiments. In embodiments, however, the skirt 142 may be positioned radially inward of the frame 138, with the frame 138 positioned between the skirt 142 and the bioresorbable skirt 132. In such embodiments, the inner skirt 140 may be excluded if desired, with the main skirt 142 utilized in its place. In embodiments, a combination of the inner skirt 140 and the main skirt 142 may be utilized as desired.

The bioresorbable skirt 132 may couple to the remainder of the implantable prosthetic valve 130, including the plurality of prosthetic valve leaflets 141, with one or more bioresorbable couplers 144. Such couplers may be configured in a similar manner as the bioresorbable couplers 112 discussed in regard to FIGS. 13-15, and may be configured to dissolve after a duration within the patient's body. The bioresorbable couplers 144 may be utilized to couple the bioresorbable skirt 132 directly to the main skirt 142, the frame 138, the inner skirt 140, or the prosthetic valve leaflets 141, or a combination of locations. The bioresorbable couplers 144 may be utilized to release the outer skirt 132 from the remaining portions of the implantable prosthetic valve 130 following a duration of implantation within the patient's body.

The main skirt 142 may be configured to be thromboresistant in embodiments. As such, a reduced possibility of tissue adhesion with the main skirt 142 may result. The main skirt 142 may be configured in a similar manner as the first level 54 discussed in regard to FIGS. 1-12, and may include similar thromboresistant properties as the first level 54. For example, the main skirt 142 may comprise a PTFE or ePTFE material (among other forms of material). The main skirt 142 may couple to the frame 138, inner skirt 140, or the prosthetic valve leaflets 141, or a combination of locations, with couplers that are non-bioresorbable (such as non-resorbable sutures or another form of coupler).

Upon implantation, the bioresorbable skirt 132 may be configured to dissolve, and may be removed due to the dissolving and/or may be replaced by tissue in its place. Such a feature may result in tissue formation around the implantable prosthetic valve 130 that may enhance sealing with the implantable prosthetic valve 130. Further, the main skirt 142 may then comprise the outer skirt of the implantable prosthetic valve 130, which may be thromboresistant to reduce the possibility of further tissue adhesion. The bioresorbable skirt 132 is configured to dissolve such that the main skirt 142 comprises an outer surface of the valve body 136 upon the bioresorbable skirt 132 dissolving. As such, upon a removal or explant procedure, the thromboresistant surface may enhance the ease of such a procedure due to the lack of tissue adhesion to the thromboresistant surface.

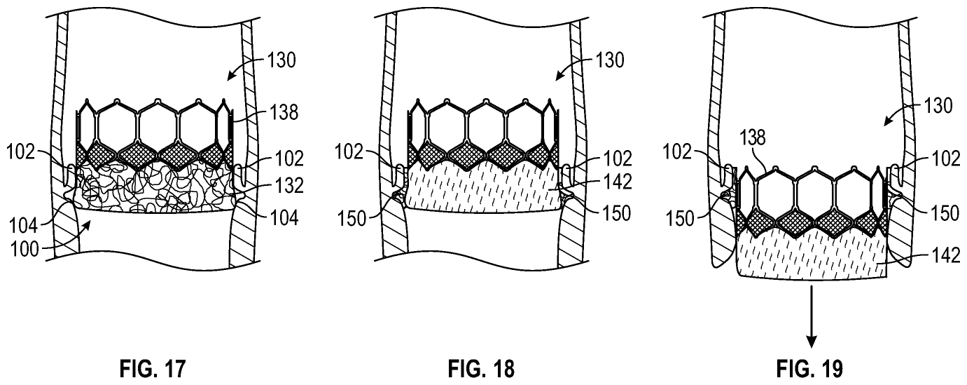

FIG. 17, for example, illustrates the implantable prosthetic valve 130 implanted within the native aortic valve 100, with the bioresorbable skirt 132 comprising the outer surface of the implantable prosthetic valve 130. The bioresorbable skirt 132 may contact the native valve leaflets 102 and/or the annulus 104 if desired.

FIG. 18 illustrates the implantable prosthetic valve 130 after a duration within the patient's body, in which the bioresorbable skirt 132 has dissolved and (as may be possible) has been replaced with tissue 150 in its place. The thromboresistant main skirt 142 is now an outer skirt of the implantable prosthetic valve 130, with tissue adhesion reduced due to the thromboresistant nature of the main skirt 142. The tissue 150 may form a uniform annular shape around the main skirt 142, which may enhance sealing around the implantable prosthetic valve 130. Such a shape of the tissue 150 may comprise an improvement over the possibly irregular shape of an implantation site, considering that any calcification of the leaflets 102 may produce an irregular sealing shape for the implantation site.

Upon removal or explant of the implantable prosthetic valve 130 being desired, a removal or explant procedure may be performed. Referring to FIG. 19, the thromboresistant nature of the main skirt 142 may enhance the ability of the implanted prosthetic valve 130 to be removed, due to a reduced tissue adhesion between the main skirt 142 and the tissue 150 and surrounding features of the patient's body. FIG. 19 illustrates the implanted prosthetic valve 130 being removed, with the main skirt 142 comprising the outer skirt of the implanted prosthetic valve 130.

Variations in the methods disclosed herein may be provided as desired. Further variations in the configurations of the implantable prosthetic valves may be provided in embodiments. For example, in embodiments, the outer skirt 132 shown in FIG. 16 may not be bioresorbable, but may be configured to allow tissue ingrowth, in a similar manner as the second level 56 shown in FIG. 1 for example. The outer skirt 132 may be constructed of materials that are configured to allow tissue ingrowth with the skirt 114, as discussed in regard to the second level 56 of the skirt 52, or may have another configuration as desired. For example, the outer skirt 132 may be made of a knit PET fabric, among other forms of materials. As such, bioresorbable couplers 144 may be configured to dissolve to release such an outer skirt 132 from the main skirt 142 during a removal or explant procedure.

The features of the prosthetic valve 130 may be implemented independently or in combination with other features disclosed herein.

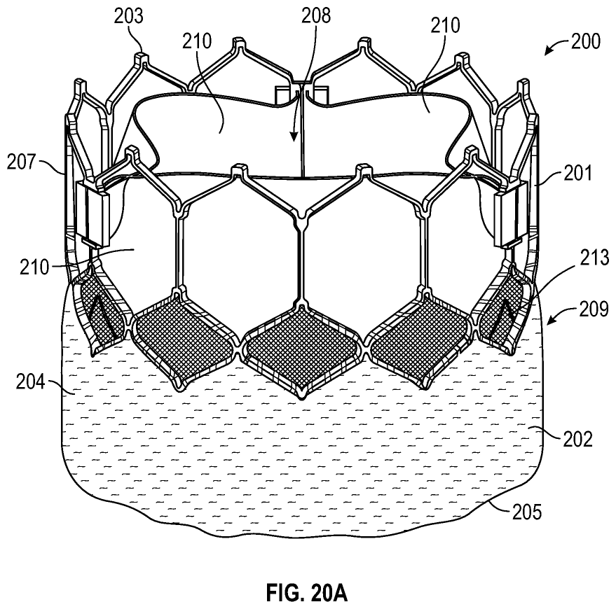

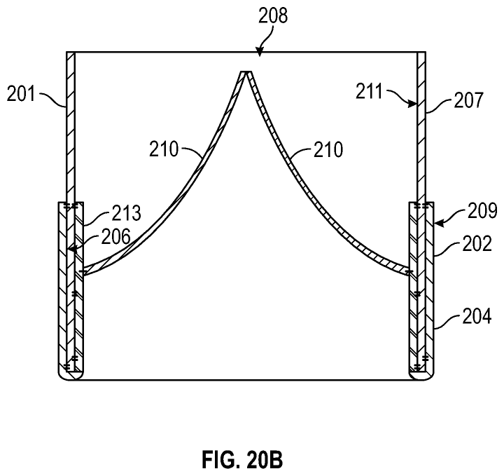

FIG. 20A illustrates a perspective view of an implant in the form of an implantable prosthetic valve 200. The implantable prosthetic valve 200 may be configured similarly as a prosthetic valve 10 as shown in FIG. 1. The prosthetic valve 200 may have a skirt 202, however, that may have a different configuration than the skirt 52 shown in FIG. 1.

The prosthetic valve 200 may include a valve body 201 that may have a proximal end 203 and a distal end 205. The proximal end 203 may comprise an outflow end of the prosthetic valve 200, and the distal end 205 may comprise an inflow end of the prosthetic valve 200. In embodiments, the prosthetic valve 200 may include a frame 207 that may be configured similarly as the frame 20 shown in FIG. 1 and may include a plurality of struts. In embodiments, the frame 207 may have a different configuration than the frame 20 shown in FIG. 1.

The valve body 201 may include an outer surface 209 and an inner surface 211 (marked in FIG. 20B) facing a flow channel 208. A plurality of prosthetic valve leaflets 210 may be positioned within the flow channel 208 and may extend inward from the inner surface 211 of the valve body 201.

The skirt 202 may have an exterior surface 204 forming at least a portion of the outer surface 209 of the valve body 201. The exterior surface 204, in embodiments, may be configured to be porous to allow tissue ingrowth within the exterior surface 204. The skirt 202, in embodiments, may include an interior surface 206 (marked in FIG. 20B) that is configured to be thromboresistant. The interior surface 206 may face towards the flow channel 208 and the prosthetic valve leaflets 210 in embodiments.

In embodiments, the skirt 202 may include an outer portion and may include an inner portion. The inner portion may be positioned radially inward of the outer portion. The outer portion, in embodiments, may comprise the exterior surface 204 of the skirt 202. The inner portion, in embodiments, may comprise the interior surface 206 of the skirt 202. Other configurations of skirts may be utilized in embodiments as desired.

Referring to FIG. 20B, the skirt 202 may comprise an outer skirt of the valve body 201 that may be positioned radially outward of the frame 207. The skirt 202 may extend circumferentially about the valve body 201 and may extend around the entirety of the valve body 201. The outer skirt may extend circumferentially about the frame 207. The valve body 201 in embodiments, may include an interior skirt 213 that may be positioned radially inward of the frame 207. The interior skirt 213 may form at least a portion of the inner surface 211 of the valve body 201.

In embodiments, the interior skirt 213 may be excluded from use and the skirt 202 may comprise the only skirt utilized with the valve body 201. The interior surface 206 of the skirt 202 may form at least a portion of the inner surface 211 of the valve body 201. In embodiments, the skirt 202 may be positioned radially inward of the frame 207 and/or may comprise the only skirt 202 utilized with the valve body 201. In embodiments, the skirt 202 may be positioned both radially outward of the frame 207 and radially inward of the frame 207.

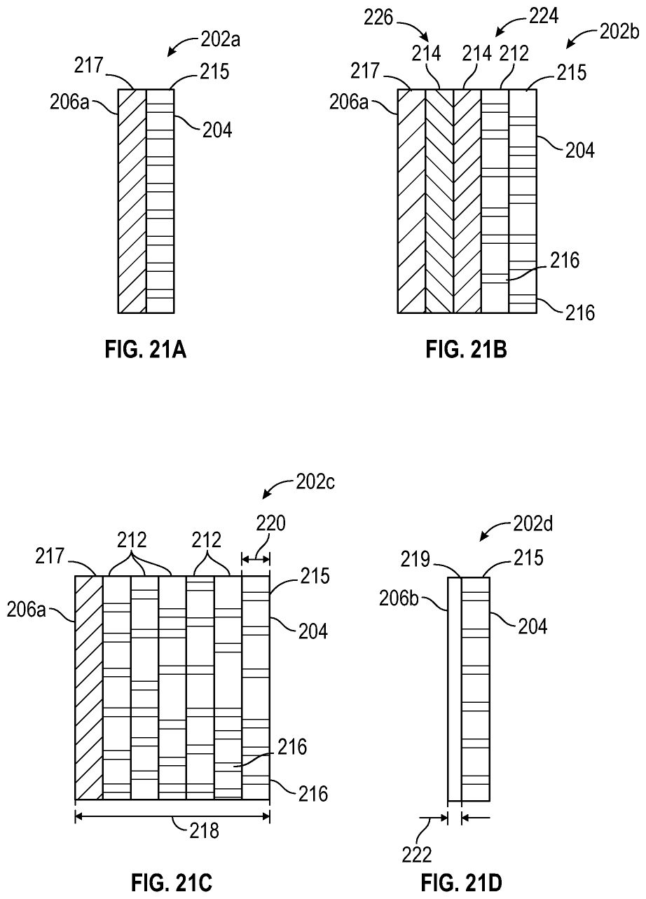

FIGS. 21A-21D illustrate a cross-sectional views of embodiments of skirts that may be utilized as the skirt 202. FIG. 21A, for example, illustrates a configurations of a skirt 202a. The skirt 202a may include a plurality of layers. The plurality of layers may be layered in a direction radially outward from the flow channel 208 shown in FIG. 20B.

The plurality of layers, in embodiments, may be laminated to each other. The lamination process may include positioning a second layer 215 upon a first layer 217 and compressing the second layer 215, which may be at a pressure and temperature. The lamination process may include positioning additional layers (e.g., a third layer, fourth layer, fifth layer, etc.) upon the layers 215, 217 and continuing to laminate at a pressure and temperature. A multi-layered structure for the skirt may result. In embodiments, a skirt may include at least two layers, at least three layers, at least four layers, at least five layers, at least six layers, or a greater number of layers as desired. In embodiments, a skirt may include seven layers, although a greater number of layers may be provided as desired.

Referring to FIG. 21A, in embodiments, a first layer 217 forming an interior surface 206a of the skirt 202a may comprise a layer of expanded polytetrafluoroethylene (ePTFE). In embodiments, the layer 217 may be compressed during a lamination process, or otherwise, with a relatively high pressure and/or temperature value to compress the layer 217 of ePTFE and reduce the porosity of the layer. The compression may reduce the porosity to seal the ePTFE such that the ePFTE layer is thromboresistant, thus resulting in a thromboresistant interior surface 206a of the skirt 202a. The compressed ePTFE layer 217 may be substantially devoid of pores and may resist tissue adhesion with the compressed ePTFE layer 217.

In embodiments, a second layer 215 may be positioned upon the compressed ePTFE layer 217. The second layer 215 may comprise an outer layer and the first layer 217 may comprise an interior layer. The second layer 215 may be compressed during a lamination process, or otherwise, with a lesser pressure and/or temperature value than the compressed ePTFE layer 217, thus leaving the second layer 215 porous and configured for tissue ingrowth with the exterior surface 204 of the skirt 202a. In embodiments, the second layer 215 may be laminated under a conventional pressure and/or temperature value for lamination of ePTFE, and the first layer 217 may be compressed under a pressure and/or temperature value that is greater than conventional values. As such, the first layer 217 and second layer 215 may be laminated to each other with the second layer 215 being porous and the first layer 217 being sealed. The exterior surface 204 of the skirt 202a may comprise a layer 215 of ePTFE that is compressed less than the compressed layer 217 of ePTFE of the interior surface 206a.

In embodiments, a greater number of layers may be provided for the skirt. FIG. 21B, for example, illustrates a cross-section view of a skirt 202b. The skirt 202b may include mid-layers 212, 214. Two mid-layers 214 and one mid-layer 212 are shown by example. In some embodiments, there may be more or less mid-layers 212, 214. The mid-layers 212, 214 may be between the exterior surface 204 and the interior surface 206a. A mid-layer 214 or a plurality of mid-layers 214 directly adjacent to the compressed ePTFE layer 217 may be a layer or layers of ePTFE that may be compressed at a same pressure and/or temperature as the compressed layer 217 of ePTFE of the interior surface 206a. As such, at least one mid-layer 214 may be sealed and thromboresistant in embodiments.

A mid-layer 212 or a plurality of mid-layers 212 directly adjacent to the layer 215 of ePTFE of the exterior surface 204 may be a layer or layers of ePTFE compressed at a same pressure and/or temperature as the layer 215 of ePTFE of the exterior surface 204. As such, the mid-layer 212 or mid-layers 212 may be porous in embodiments and configured to allow tissue ingrowth therein. In embodiments, at least one mid-layer 212 between the interior surface 206a and the exterior surface 204 may comprise porous ePTFE. In some embodiments, mid-layer 212 or mid-layers 212 may be a layer or layers of ePTFE compressed at a lower pressure and/or lower temperature than the layer 217 of ePTFE of the interior surface 206a but a higher pressure and/or higher temperature than the layer 215 of ePTFE of the exterior surface 204. For example, as shown in FIG. 21B, the mid-layer 212 may include fewer pores 216 than the exterior surface 204 due to being compressed at a higher pressure and/or higher temperature than the layer 215 of ePTFE of the exterior surface 204.

FIG. 21C illustrates a cross-section view of a skirt 202c, which may include seven layers. The skirt 202c may have a thickness 218. In embodiments, the thickness 218 may be between 50 to 60 micrometers (μm), although greater or lesser thicknesses may be provided in embodiments as desired. Each layer may have a thickness 220. In embodiments, the thickness 220 may be between 5 to 10 μm, although a greater or lesser thickness may be provided in embodiments as desired.

The skirt 202c may be resistant to tears due to the thickness 218 of the skirt 202c. The skirt 202c may include multiple mid-layers 212. As shown in FIG. 21C, the skirt 202c may include five mid-layers 212, for example. The mid-layers 212 may have varying degrees of porosity. For example, the porosity of the mid-layers 212 may be the same from a direction of the interior surface 206a to the exterior surface 204. The porosity of the mid-layers 212 may be the same as the porosity of the outer layer 215 in embodiments, or may be different. As such, each mid-layer 212 or some mid-layers 212 may be compressed at a same pressure and/or a same temperature than the outer layer 215. In embodiments, the porosity of the mid-layers 212 may increase from a direction of the interior surface 206a to the exterior surface 204. As such, each mid-layer 212 or some mid-layers 212 may be compressed at a lower pressure and/or a lower temperature than the preceding mid-layer 212 or mid-layers 212 in the direction of the interior surface 206a to the exterior surface 204. There may be tissue ingrowth within one or more mid-layers 212.

In embodiments, the pores 216 of the layer 215 of the exterior surface 204 and the pores 216 of the mid-layers 212 may be aligned or misaligned. The pores 216 of the mid-layers 212 may be aligned or misaligned with the pores 216 of other mid-layers 212.

FIG. 21D illustrates a cross-section view of a skirt 202d, in which the interior surface 206b may comprise a layer 219 of sealed polytetrafluoroethylene (PTFE). The layer 219 may comprise an interior layer. The layer 219 of the interior surface 206b may have a thickness that is less than an outer layer 215 of the skirt 202d forming the exterior surface 204 of the skirt 202d. For example, a thickness 222 of the layer 219 of the interior surface 206b may be 1 μm, and a thickness of the layer 215 forming the exterior surface 204 may be greater than 1 μm (e.g., 5 to 10 μm). In embodiments, other ranges of thickness may be provided as desired.

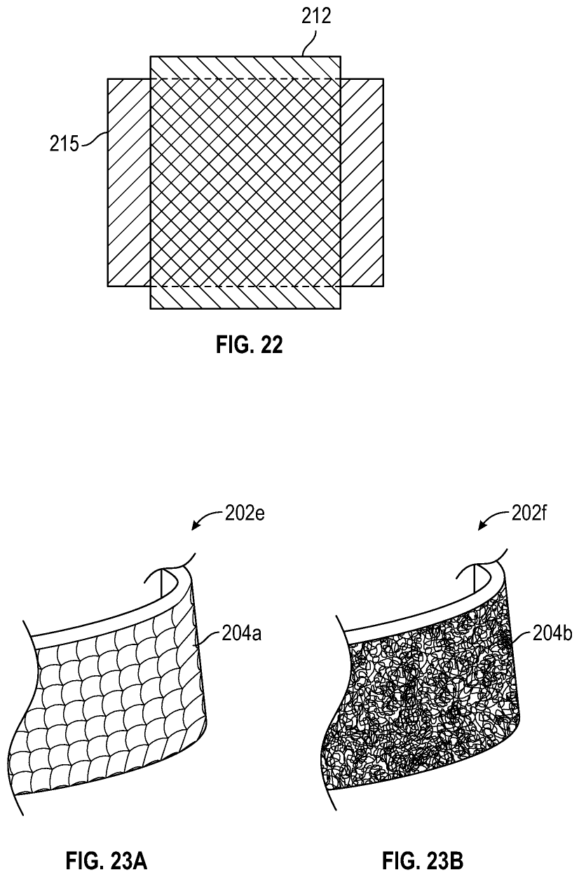

The layers of the skirts disclosed herein may be compressed to each other in a lamination process. In embodiments, layers of the skirts disclosed herein may be laminated over each other at right-angles relative to each other. FIG. 22, for example illustrates a front schematic view of layers 215, 212 of a skirt 202b of the implantable prosthetic valve. The layers 215, 212 may be ePTFE layers that may be oriented at right-angles relative to each other. Additional layers may be utilized in embodiments and oriented at right-angles relative to adjacent layers of the skirt. Other configurations of skirts may be utilized in embodiments as desired.

FIG. 23A illustrates a partial isolated perspective view of a skirt 202e that may be utilized with the implantable prosthetic valve 200 (see FIG. 20A). The skirt 202e may include features as disclosed herein regarding FIGS. 20A-22. In embodiments, an exterior surface 204a of the skirt 202e may be a textured surface. The textured surface may further allow tissue ingrowth within the skirt 202e. The textured surface may include bumps or protrusions. In some embodiments, only a portion of the exterior surface 204a may be a textured surface.

FIG. 23B illustrates a partial isolated perspective view of a skirt 202f that may be utilized with the implantable prosthetic valve 200 (see FIG. 20A). The skirt 202f may include features as disclosed herein regarding FIGS. 20A-23A. In embodiments, an exterior surface 204b of the skirt 202f may include filaments extending therefrom. The filaments may further allow tissue ingrowth within the skirt 202f. In some embodiments, only a portion of the exterior surface 204b may include filaments.

In embodiments, other configurations of skirts may be utilized. For example, in embodiments, the skirt may include a single layer. In embodiments, the layers may be compressed and coupled to each other in a process other than lamination. For example, compressed layers may be sutured together or otherwise coupled to each other. The skirts as disclosed herein may be coupled to a frame in a variety of manners, including bonding or stitching. For example, sutures may couple the skirts to a frame as desired.

In embodiments, a porous outer surface of the skirt may allow for tissue ingrowth that may reduce the possibility of leakage outside of the valve (e.g., paravalvular leakage). The tissue ingrowth may serve to fill spaces that may exist between the valve and an irregular shaped valve annulus. The tissue ingrowth may further serve to anchor the valve to a desired implantation site. A thromboresistant interior surface of the skirt may serve to reduce the possibility of tissue ingrowth that may reach the flow channel 208 and/or the leaflets 210 and possibly reduce operation of the prosthetic valve. The possibility of overgrowth may be reduced via use of a thromboresistant interior surface.

In embodiments, the use of ePTFE may provide enhanced tear resistance for the prosthetic valve. Porous ePTFE may further allow for a desired tissue ingrowth with the skirt.

The porous ePTFE in embodiments, may be positioned on an outer portion of the skirt. For example, referring to FIG. 21B, an outer portion 224 of the skirt 202b may comprise the mid-layer 212 and the outer layer 215. The outer portion 224, in embodiments, may include the exterior surface 204 of the skirt 202b or another part of the skirt 202b as desired. The outer portion 224 in embodiments may include one or more layers of porous ePTFE. The skirt 202b may include an inner portion 226 that is positioned radially inward of the outer portion 224. The inner portion 226 may include the interior layer 217 and the mid-layers 214. The inner portion 226 may be thromboresistant. The inner portion 226 may include one or more sealed layers, which may include compressed ePTFE as shown in FIG. 21B, or may include a layer 219 of sealed PTFE as shown in FIG. 21 D for example. The inner portion 226 may include the interior surface 206a of the skirt 202b in embodiments or another part of the skirt 202b as desired. Other configurations of layers (e.g., layers as described in regard to FIGS. 21A-23B) may comprise outer and inner portions as desired. Other configurations of skirts and inner portions and outer portions of skirts may be utilized as desired.

In embodiments, other porous materials than ePTFE may be utilized. Other thromboresistant materials may be utilized in embodiments as desired.

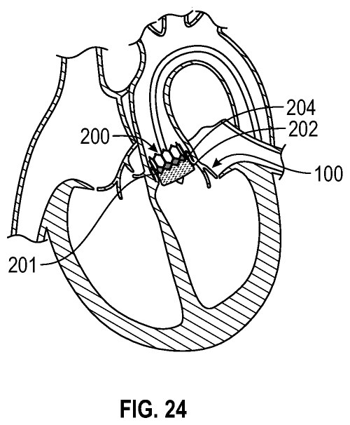

FIG. 24 illustrates a schematic view of the implantable prosthetic valve 200 implanted at a native aortic valve 100. The prosthetic valve 200 may be implanted utilizing methods disclosed herein or other methods as desired. The valve body 201 may be configured to be implanted to a native heart valve, and the exterior surface of the skirt 202 may be configured to allow tissue ingrowth with the native leaflets of the native heart valve and/or the native valve annulus. In embodiments, the force of an exterior surface 204 of the valve body 201 against the native valve leaflets and the native valve annulus may anchor the implantable prosthetic valve 200 to the native aortic valve 100. Notably, the skirt 202 may be positioned such that the exterior surface 204 contacts the native valve leaflets and/or the native valve annulus.

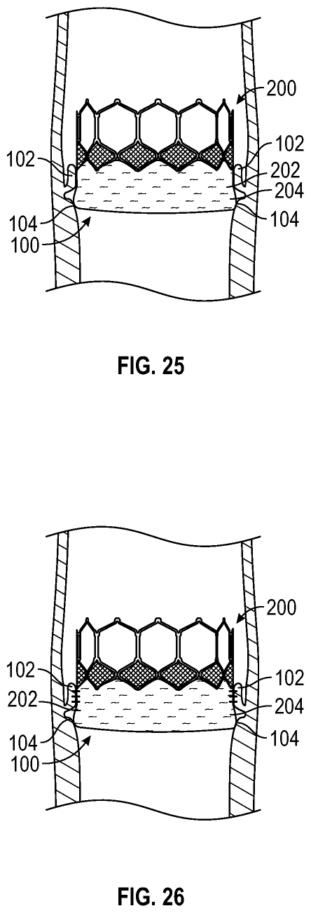

FIG. 25 illustrates a schematic view of the implantable prosthetic valve 200 implanted at the native aortic valve 100. The exterior surface 204 is shown to contact one or more native valve leaflets 102 and the native valve annulus 104. The porous exterior surface 204 may allow for tissue ingrowth, which may occur with the native valve leaflets 102 and the native valve annulus 104. Such tissue ingrowth may comprise neointimal tissue.

FIG. 26 illustrates a schematic view of the implantable prosthetic valve 200 having been implanted for a duration, which has resulted in tissue adhesion between the exterior surface 204 and the native valve leaflets 102 and the native valve annulus 104. The tissue adhesion may improve anchoring and sealing of the implantable prosthetic valve 200. Paravalvular leakage may be reduced. In some embodiments, the tissue adhesion may only be with the native valve leaflets 102.

The features of the prosthetic valve 200 and skirts disclosed herein may be implemented independently or in combination with other features disclosed herein. For example, the features of the prosthetic valve 200 and skirts may be utilized with the features of the embodiments of FIGS. 1-19 as desired.

As discussed, various forms of implants may be utilized with the embodiments disclosed herein, including prosthetic heart valves, or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state. A crimping device as disclosed herein may assist in moving the implant to the compressed or undeployed state.

The deployment apparatuses as disclosed herein may be utilized for aortic, mitral, tricuspid, and pulmonary replacement and repair as well. The deployment apparatuses may comprise deployment apparatuses for delivery of other forms of implants, such as stents or filters, or diagnostic devices, among others.

The deployment apparatuses and the systems disclosed herein may be used in transcatheter aortic valve implantation (TAVI) or replacement of other native heart valves (e.g., mitral, tricuspid, or pulmonary). The deployment apparatuses and the systems disclosed herein may be utilized for transarterial access, including transfemoral access, to a patient's heart. The deployment apparatuses and systems may be utilized in transcatheter percutaneous procedures, including transarterial procedures, which may be transfemoral or transjugular. Transapical procedures, among others, may also be utilized. Other procedures may be utilized as desired.

Features of embodiments may be modified, substituted, excluded, or combined across embodiments as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

The features of the embodiments disclosed herein may be implemented independently, or independent of other components disclosed herein. The various apparatuses of the system may be implemented independently.

As a first example, an implantable prosthetic valve. The implantable prosthetic valve may include a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level; and a plurality of prosthetic valve leaflets positioned within the flow channel and extending inward from the inner surface of the valve body.

Implementations of the first example may include one or more of the following. The distal end may be an inflow end of the valve body, and the first level may be adjacent to the distal end. The proximal end may be an outflow end of the valve body, and the second level may be positioned between the first level and the proximal end. The second level may be positioned adjacent to the first level in an axial direction. The second level may be coupled to the first level by bonding or stitching. The first level and the second level may both extend circumferentially about the valve body. The valve body may include a frame, and the first level and the second level may both be positioned radially outward of the frame. The first level and the second level may both be coupled to the frame by bonding or stitching. A ratio of relative axial extent along the valve body of the first level to the second level may be 1:1 or greater. The first level may comprise a first material and the second level may comprise a second material that is different than the first material. The first material may include at least one or more of a polytetrafluoroethylene, an ultra high molecular weight polyethylene, or a coated thermoplastic polyurethane. The second material may include polyethylene terephthalate. The first level may include a thromboresistant coating. The second level may include textured yarns extending radially outward, and the first level may include a smooth texture configured to inhibit tissue growth. The valve body may be configured to be implanted to a native heart valve, and the second level may be configured to allow tissue ingrowth with native leaflets of the native heart valve, and the first level may be configured to be positioned within an annulus of the native heart valve.

As a second example, a method. The method may comprise implanting a prosthetic valve within a patient's body, the prosthetic valve including: a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level, and a plurality of prosthetic valve leaflets positioned within the flow channel and extending inward from the inner surface of the valve body.

Implementations of the second example may include one or more of the following. The method may further comprise implanting the prosthetic valve to a native heart valve. The method may further comprise contacting the second level to a native leaflet of the native heart valve. The method may further comprise positioning the first level at an annulus of the native heart valve. The distal end may be an inflow end of the valve body, and the proximal end may be an outflow end of the valve body, and the second level may be positioned between the first level and the proximal end of the valve body. The first level and the second level may both extend circumferentially about the valve body. The first level may comprise a first material and the second level may comprise a second material that is different than the first material. The first material may include at least one or more of a polytetrafluoroethylene, an ultra high molecular weight polyethylene, or a coated thermoplastic polyurethane. The second material may include polyethylene terephthalate. The first level may include a thromboresistant coating.

As a third example, a method. The method may comprise removing an implanted prosthetic valve from a location within a patient's body, the implanted prosthetic valve including: a valve body having a proximal end, a distal end, an outer surface, and an inner surface facing a flow channel, and a skirt including a first level forming at least a portion of the outer surface of the valve body and configured to be thromboresistant and a second level forming at least a portion of the outer surface of the valve body and configured to allow tissue ingrowth with the second level, and a plurality of prosthetic valve leaflets positioned within the flow channel and extending inward from the inner surface of the valve body.

Implementations of the third example may include one or more of the following. The method may comprise removing the implanted prosthetic valve from a native heart valve. The first level may be positioned at an annulus of the native heart valve. The method may include severing a native leaflet from the patient's body, the native leaflet being in contact with the second level of the skirt. The native leaflet may be bonded to the second level of the skirt, and the method further comprises removing the native leaflet and the second level of the skirt from the patient's body. The distal end may be an inflow end of the valve body, and the proximal end may be an outflow end of the valve body, and the second level may be positioned between the first level and the proximal end of the valve body. The first level and the second level may both extend circumferentially about the valve body. The first level may comprise a first material and the second level may comprise a second material that is different than the first material. The first material may include at least one or more of a polytetrafluoroethylene, an ultra high molecular weight polyethylene, or a coated thermoplastic polyurethane. The second material may include polyethylene terephthalate.

As a fourth example, an implantable prosthetic valve. The implantable prosthetic valve may include a plurality of prosthetic valve leaflets; and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

Implementations of the fourth example may include one or more of the following. The valve body may include a frame and the skirt may be positioned radially outward of the frame and may be coupled to the frame with the one or more bioresorbable couplers. The skirt may be a first skirt, and the valve body may include a second skirt positioned radially inward of the frame, and the first skirt may be coupled to the second skirt with the one or more bioresorbable couplers. The skirt may be coupled to a scallop line of the prosthetic valve leaflets with the one or more bioresorbable couplers. The skirt may be configured to allow tissue ingrowth with the skirt. The skirt may be a bioresorbable skirt. The skirt may be a first skirt positioned radially outward of a second skirt, with the second skirt configured to be thromboresistant. The valve body may include a frame, and the second skirt may be positioned radially outward of the frame. The one or more bioresorbable couplers may include one or more bioresorbable sutures. The skirt may be configured to separate from the plurality of prosthetic valve leaflets upon dissolving of the one or more bioresorbable couplers.

As a fifth example, an implantable prosthetic valve. The implantable prosthetic valve may include a valve body having a proximal end, a distal end, an outer surface, and an inner surface facing a flow channel, and a first skirt forming at least a portion of the outer surface of the valve body and configured to be bioresorbable, and a second skirt positioned radially inward of the first skirt and configured to be thromboresistant; and a plurality of prosthetic valve leaflets positioned within the flow channel and extending inward from the inner surface of the valve body.

Implementations of the fifth example may include one or more of the following. The valve body may comprise a frame. The first skirt and the second skirt may be positioned radially outward of the frame. The first skirt may be coupled to the plurality of prosthetic valve leaflets with one or more bioresorbable couplers. The one or more bioresorbable couplers may include one or more bioresorbable sutures. The first skirt may be coupled to the plurality of prosthetic valve leaflets with one or more non-bioresorbable couplers. The first skirt may be configured to dissolve such that the second skirt comprises an outer surface of the valve body upon the first skirt dissolving. The first skirt and the second skirt may both extend circumferentially about the valve body. The second skirt may comprise polytetrafluoroethylene (PTFE) or expanded polytetrafluoroethylene (ePTFE). The valve body may be configured to be deployed to an aortic valve.

As a sixth example, a method. The method may include implanting a prosthetic valve within a patient's body. The prosthetic valve may include a plurality of prosthetic valve leaflets, and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

Implementations of the sixth example may include one or more of the following. The method may further comprise implanting the prosthetic valve to a native heart valve. The method may further comprise contacting the skirt to a native leaflet of the native heart valve. The one or more bioresorbable couplers may include one or more bioresorbable sutures. The skirt may be configured to separate from the plurality of prosthetic valve leaflets upon dissolving of the one or more bioresorbable couplers. The valve body may include a frame and the skirt is positioned radially outward of the frame and is coupled to the frame with the one or more bioresorbable couplers. The skirt may be configured to allow tissue ingrowth with the skirt. The skirt may be a bioresorbable skirt. The skirt may be a first skirt positioned radially outward of a second skirt, with the second skirt configured to be thromboresistant. The valve body may include a frame, and the second skirt may be positioned radially outward of the frame.

As a seventh example, a method. The method may include removing an implanted prosthetic valve from a location within a patient's body, the implanted prosthetic valve upon implantation including: a plurality of prosthetic valve leaflets, and a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

Implementations of the seventh example may include one or more of the following. The method may further comprise removing the implanted prosthetic valve from a native heart valve. The method may further comprise separating the skirt from the plurality of prosthetic valve leaflets while removing the implanted prosthetic valve from the native heart valve. The one or more bioresorbable couplers may have dissolved. The method may further comprise removing the implanted prosthetic valve from the location within the patient's body without the skirt. Upon implantation, the valve body may include a frame and the skirt may be positioned radially outward of the frame and may be coupled to the frame with the one or more bioresorbable couplers. The skirt may be configured to allow tissue ingrowth with the skirt. The skirt may be a bioresorbable skirt. Upon implantation, the skirt may be a first skirt positioned radially outward of a second skirt, with the second skirt configured to be thromboresistant. The valve body may include a frame, and the second skirt may be positioned radially outward of the frame.

As an eighth example, an implantable prosthetic valve. The implantable prosthetic valve may include a valve body having an outer surface and an inner surface facing a flow channel, and a skirt including an exterior surface forming at least a portion of the outer surface of the valve body and configured to be porous to allow tissue ingrowth within the exterior surface and an interior surface that is configured to be thromboresistant; and a plurality of prosthetic valve leaflets positioned within the flow channel and extending inward from the inner surface of the valve body.

Implementations of the eighth example may include one or more of the following. The skirt may include a plurality of layers. The plurality of layers may be layered in a direction radially outward from the flow channel. The plurality of layers may be laminated to each other. The plurality of layers may include at least three layers. The plurality of layers may include expanded polytetrafluoroethylene (ePTFE). The plurality of layers may comprise layers of ePTFE oriented at right-angles relative to each other. The interior surface may comprise a compressed layer of ePTFE. The interior surface may be sealed. The exterior surface may comprise a layer of ePTFE that is less compressed than the compressed layer of ePTFE of the interior surface. The layer of ePTFE of the exterior surface may be compressed at a lower pressure or a lower temperature than the compressed layer of ePTFE of the interior surface. At least one mid-layer between the interior surface and the exterior surface directly adjacent to the compressed layer of ePTFE of the interior surface may be compressed at a same pressure and a same temperature as the compressed layer of ePTFE of the interior surface. The interior surface may comprise a layer of sealed polytetrafluoroethylene (PTFE). The layer of sealed PTFE may have a thickness that is less than a layer of the skirt forming the exterior surface of the skirt. At least one mid-layer between the interior surface and the exterior surface may comprise porous ePFTE. At least a portion of the exterior surface may be a textured surface or includes filaments extending from the exterior surface to further allow tissue ingrowth. The skirt may extend circumferentially about the valve body. The valve body may include a frame, and the skirt may be positioned radially outward of the frame. The skirt may be coupled to the frame by bonding or stitching. The valve body may be configured to be implanted to a native heart valve, and the exterior surface of the skirt may be configured to allow tissue ingrowth with native leaflets of the native heart valve.

As a ninth example, a method. The method may comprise implanting a prosthetic valve within a patient's body, the prosthetic valve including: a valve body having an outer surface and an inner surface facing a flow channel, and a skirt including an exterior surface forming at least a portion of the outer surface of the valve body and configured to be porous to allow tissue ingrowth within the exterior surface and an interior surface that is configured to be thromboresistant; and a plurality of prosthetic valve leaflets positioned within the flow channel and extending inward from the inner surface of the valve body.

Implementations of the ninth example may include one or more of the following. The method may further comprise implanting the prosthetic valve to a native heart valve. The method may further comprise contacting the exterior surface of the skirt to a native leaflet or an annulus of the native heart valve. The skirt may include a plurality of layers. The plurality of layers may be layered in a direction radially outward from the flow channel. The plurality of layers may be laminated to each other. The plurality of layers may include expanded polytetrafluoroethylene (ePTFE). The interior surface may comprise a compressed layer of ePTFE. The interior surface may comprise a layer of sealed polytetrafluoroethylene (PTFE). At least one mid-layer between the interior surface and the exterior surface may comprise porous ePFTE.

As a tenth example, an implantable prosthetic valve. The implantable prosthetic valve may include a valve body having an outer surface and an inner surface facing a flow channel, and a skirt including porous expanded polytetrafluoroethylene (ePTFE) to allow tissue ingrowth within the skirt; and a plurality of prosthetic valve leaflets positioned within the flow channel and extending inward from the inner surface of the valve body.

Implementations of the tenth example may include one or more of the following. The skirt may include a plurality of layers of ePTFE laminated to each other, the plurality of layers being layered in a direction radially outward from the flow channel. An outer portion of the skirt may include the porous ePTFE. The skirt may include an inner portion positioned radially inward of an outer portion of the skirt and is configured to be thromboresistant. The inner portion may include a layer of compressed ePTFE. The outer portion of the skirt may include a layer of ePTFE that is compressed less than the layer of compressed ePTFE of the inner portion. The inner portion may include a layer of sealed polytetrafluoroethylene (PTFE). The outer portion may form an exterior surface of the skirt and the inner portion may form an interior surface of the skirt. The skirt may include an outer layer, an interior layer, and at least one mid-layer, with the outer layer and the at least one mid-layer may comprise porous ePTFE and the interior layer may be sealed. The valve body may include a frame, and the skirt may extend circumferentially about the frame and may be positioned radially outward of the frame.

As an eleventh example, a method. The method may comprise implanting a prosthetic valve within a patient's body, the prosthetic valve including: a valve body having an outer surface and an inner surface facing a flow channel, and a skirt including porous expanded polytetrafluoroethylene (ePTFE) to allow tissue ingrowth within the skirt; and a plurality of prosthetic valve leaflets positioned within the flow channel and extending inward from the inner surface of the valve body.

Implementations of the eleventh example may include one or more of the following. The method may further comprise implanting the prosthetic valve to a native heart valve. The method may further comprise contacting the porous expanded polytetrafluoroethylene (ePTFE) to a native leaflet or an annulus of the native heart valve. The skirt may include a plurality of layers of ePTFE laminated to each other, the plurality of layers being layered in a direction radially outward from the flow channel. The skirt may include an inner portion configured to be thromboresistant. The inner portion may include a layer of compressed ePTFE. The inner portion may include a layer of sealed polytetrafluoroethylene (PTFE). An outer portion of the skirt may include the porous ePTFE. The skirt may include an outer layer, an interior layer, and at least one mid-layer, with the outer layer and the at least one mid-layer comprising porous ePTFE and the interior layer being sealed. The valve body may include a frame, and the skirt may extend circumferentially about the frame and may be positioned radially outward of the frame.

Any of the features of any of the examples, including but not limited to any of the first through eleventh examples referred to above, is applicable to all other aspects and embodiments identified herein, including but not limited to any embodiments of any of the first through eleventh examples referred to above. Moreover, any of the features of an embodiment of the various examples, including but not limited to any embodiments of any of the first through eleventh aspects referred to above, is independently combinable, partly or wholly with other examples described herein in any way, e.g., one, two, or three or more examples may be combinable in whole or in part. Further, any of the features of the various examples, including but not limited to any embodiments of any of the first through eleventh examples referred to above, may be made optional to other examples. Any example of a method can be performed by a system or apparatus of another example, and any aspect or embodiment of a system or apparatus can be configured to perform a method of another aspect or embodiment, including but not limited to any embodiments of any of the first through eleventh examples referred to above.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An implantable prosthetic valve comprising:
a valve body having a proximal end, a distal end, an outer surface, and an inner surface facing a flow channel, and a skirt including a first level forming at least a portion at the outer surface of the valve body and configured to be thromboresistant and a second level forming at least a portion of the outer surface of the valve body and configured to allow tissue ingrowth with the second level; and
a plurality of prosthetic valve leaflets positioned within the flow channel and extending inward from the inner surface of the valve body.

2. The implantable prosthetic valve of claim 1, wherein the distal end is an inflow end of the valve body, and the first level is adjacent to the distal end.

3. The implantable prosthetic valve of claim 1, wherein the proximal end is an outflow end of the valve body, and the second level is positioned between the first level and the proximal end.

4. The implantable prosthetic valve of claim 1, wherein the second level is positioned adjacent to the first level in an axial direction.

5. The implantable prosthetic valve of claim 1, wherein the second level is coupled to the first level by bonding or stitching.

6. The implantable prosthetic valve of claim 1, wherein the first level and the second level both extend circumferentially about the valve body.

7. The implantable prosthetic valve of claim 1, wherein the valve body includes a frame, and the first level and the second level are both positioned radially outward of the frame.

8. The implantable prosthetic valve of claim 7, wherein the first level and the second level are both coupled to the frame by bonding or stitching.

9. The implantable prosthetic valve of claim 1, wherein a ratio of relative axial extent along the valve body of the first level to the second level is 1:1 or greater.

10. The implantable prosthetic valve of claim 1, wherein the first level comprises a first material and the second level comprises a second material that is different than the first material.

11. The implantable prosthetic valve of claim 10, wherein the first material includes at least one or more of a polytetrafluoroethylene, an ultra high molecular weight polyethylene, or a coated thermoplastic polyurethane.

12. The implantable prosthetic valve of claim 1, wherein the second level includes textured yarns extending radially outward, and the first level includes a smooth texture configured to inhibit tissue growth.

13. The implantable prosthetic valve of claim 1, wherein the valve body is configured to be implanted to a native heart valve, and the second level is configured to allow tissue ingrowth with native leaflets of the native heart valve, and the first level is configured to be positioned within an annulus of the native heart valve.

14. An implantable prosthetic valve comprising:
a plurality of prosthetic valve leaflets; and
a valve body having a proximal end, a distal end, an outer surface, and an inner surface facing a flow channel in which the plurality of prosthetic valve leaflets are positioned, and a skirt forming at least a portion of the outer surface of the valve body and being coupled to the plurality of prosthetic valve leaflets with one or more bioresorbable couplers.

15. The implantable prosthetic valve of claim 14, wherein the valve body includes a frame and the skirt is positioned radially outward of the frame and is coupled to the frame with the one or more bioresorbable couplers.

16. The implantable prosthetic valve of claim 15, wherein the skirt is a first skirt, and the valve body includes a second skirt positioned radially inward of the frame, and the first skirt is coupled to the second skirt with the one or more bioresorbable couplers.

17. The implantable prosthetic valve of claim 14, wherein the skirt is coupled to a scallop line of the prosthetic valve leaflets with the one or more bioresorbable couplers.

18. An implantable prosthetic valve comprising:
a valve body having an outer surface and an inner surface facing a flow channel, and a skirt including an exterior surface forming at least a portion of the outer surface of the valve body and configured to be porous to allow tissue ingrowth within the exterior surface and an interior surface that is configured to be thromboresistant; and
a plurality of prosthetic valve leaflets positioned within the flow channel and extending inward from the inner surface of the valve body;
wherein the skirt includes a plurality of layers.

19. The implantable prosthetic valve of claim 18, wherein the plurality of layers are layered in a direction radially outward from the flow channel.

20. The implantable prosthetic valve of claim 18, wherein the plurality of layers are laminated to each other.

21. The implantable prosthetic valve of claim 18, wherein the plurality of layers include at least three layers.

22. The implantable prosthetic valve of claim 18, wherein the plurality of layers comprise layers of ePTFE oriented at right-angles relative to each other.
